(19) United States
(12) Patent Application Publication
(10) Pub. No.: US 2019/0192527 A1
(43) Pub. Date: Jun. 27, 2019

(54) COMPOSITIONS COMPRISING PIKFYVE INHIBITORS AND METHODS RELATED TO INHIBITION OF RANK SIGNALING

(71) Applicant: AI Therapeutics, Inc., Guilford, CT (US)

(72) Inventors: Sophia Gayle, East Haven, CT (US); Jonathan M. Rothberg, Guilford, CT (US); Henri Lichenstein, Guilford, CT (US)

(21) Appl. No.: 16/327,643

(22) PCT Filed: Aug. 17, 2017
(86) PCT No.: PCT/US2017/047264

§ 371 Date: Feb. 22, 2019

Related U.S. Application Data
(60) Provisional application No. 62/379,330, filed on Aug. 25, 2016.

(51) Int. Cl. A61K 31/5377; A61P 19/08; A61P 35/04

(52) U.S. Cl. CPC A61K 31/5377; A61P 35/04; A61P 19/08

(57) ABSTRACT

The present invention relates to the use of PIKfyve inhibitors to inhibit RANKL/RANK signaling and related compositions and methods.

### CROSS REFERENCE TO RELATED APPLICATIONS

This application claims the benefit of priority from U.S. Provisional Application Ser. No. 62/379,330, filed on Aug. 25, 2016, the content of which is hereby incorporated by reference in its entirety.

### FIELD OF THE INVENTION

The present invention relates to compositions for inhibiting RANKL/RANK signaling and related therapeutic methods.

### BACKGROUND OF THE INVENTION

The bone remodeling cycle maintains the integrity of the skeleton through the activities of bone-forming osteoblasts, which synthesize and mineralize bone matrix, and bone-degrading osteoclasts, which dissolve bone and enzymatically degrade extracellular matrix proteins (Teitelbaum S L et al. Nature Reviews Genetics. 2003; 4(8):638-649 ( ). Normal osteoclast activity is necessary for bone growth and remodeling and maintenance of calcium and phosphate ion homeostasis.

Osteoclasts are unique multinucleated cells within bone that are responsible for bone degradation and resorption. These are the only cells in the body known to be capable of this function. These cells are derived from mononuclear precursors that are the progeny of stem-cell populations located in the bone marrow, spleen, and liver. Proliferation of these stem-cell populations produces osteoclastic precursors, which migrate via vascular routes to skeletal sites. These cells then differentiate and fuse with each other to form osteoclasts, or alternatively, fuse with existing osteoclasts.

Inappropriately increased osteoclast activity can result in decreased bone mass due to a remodeling cycle favoring bone resorption. Inappropriate bone loss is a result or complication of a number of different diseases and disorders, including multiple myeloma, osteoporosis, rheumatoid arthritis, periodontal disease, Paget's disease, familial expansile osteolysis, and expansile skeletal hyperphosphatasia.

In addition, bone loss may be associated with cancer, including solid tumors and metastatic solid tumors. For example, bone loss may be associated with breast cancer, prostate cancer, thyroid cancer, kidney cancer, lung cancer, esophageal cancer, rectal cancer, bladder cancer, cervical cancer, ovarian cancer, and liver cancer, and gastrointestinal tract cancer.

Osteoclast precursors originate from monocyte/macrophage lineage hematopoietic cells within the bone marrow and blood stream. Receptor activator of nuclear factor kB ligand (RANKL) and macrophage colony stimulating factor (M-CSF) are required for the differentiation of these precursors into osteoclasts (Teitelbaum S L. Science. 2000 289:1504-1508). RANKL is produced by osteoblasts and activated B and T cells and is required for the fusion and differentiation of osteoclast precursors into large multinucleated osteoclasts. RANKL also plays an additional role in the activation and survival of mature osteoclasts (Jimi E., et al., J Immunol. 1999.163:434-442). Osteoclast differentiation is induced by RANKL binding to receptor activator of nuclear factor kB (RANK) present on osteoclast precursors.

Inflammatory cytokines such as TNFα, IL-1, IL-6, IL-17 and IL-23 enhance osteoclast differentiation by inducing RANKL expression in osteoblasts and RANK receptor expression in myeloid precursor cells (Chen L et al. Eur. J Immunol. 2008 38(10):2845-54). Exposure to IL-23 in vivo is associated with increased osteoclast differentiation, severe systemic bone loss as well as chronic arthritis. Osteoclast precursors derived from IL-23p19 null mice have defective osteoclast differentiation and function (Adamopoulos I E et al. J Immunol. 2011187(2):951-9). The inflammatory cytokine IL-12 plays a dual role in osteoclast differentiation, both enhancing osteoclast differentiation by inducing Th1 cytokines and repressing osteoclast differentiation by resulting in the degradation of the RANK adaptor TRAF6 (Queiroz-Junior C M et al. Clin Dev Immunol. 2010: 327417). In addition to IL-12, the cytokine IL-10 also inhibits osteoclast differentiation by inhibiting RANKL-induced NFATc1 expression and translocation into the nucleus (Evans K E et al. BMC Cell Biol. 2007 8:4).

RANKL/RANK signaling has also been implicated in cancer progression and metastasis. High RANK levels are associated with progression in breast and renal cancer (Palafox M et al. Cancer Res. 2012 72(11):2879-88; Santini D et al. PLoS One. 2011 6(4):e19234; Mikami S et al. J Pathol. 2009 218(4):530-539). T-cell derived RANKL promotes metastasis of breast cancer cells in mice and has been implicated in metastasis in a prostate cancer model (Tan W. Nature. 2011 470:548-553; Luo J L et al. Nature. 2007 446(7136):690-694). The anti-RANKL antibody denosumab demonstrated synergistic activity in combination with the anti-CTLA4 antibody ipilimumab against cancer metastases, both in a clinical case study and in a preclinical mouse model (Smyth M J et al. J Clin Oncol. 2016 34(12):e104-6). Blocking RANKL interaction with RANK inhibited osteroclastic bone resorption and myeloma tumor burden in myeloma (Heath D J et al. Cancer Res. 2007 67(1): 202-8; Weibaecher K N et al. Nat Rev Cancer. 2011 11(6):411-425).

Apilimod is an immunomodulatory small molecule that was first identified as an inhibitor of TLR-induced IL-12 and IL-23 cytokine production and later evaluated for the inflammatory and auto-immune indications of Crohn's disease, psoriasis, and rheumatoid arthritis (Cai X et al. Chem Biol. 2013; 20(7):912-21: Krausz S et al. Arthritis Rheum. 2012 64(6):1750-5; Sands B E et al. Inflamm Bowel Dis. 2010 16(7):1209-18; Wada Y et al. PLoS One. 2012 7(4):e35069). Apilimod has been demonstrated to inhibit the production of a range of osteogenic cytokines, including IL-12, IL-23, and TNFα, in addition to promoting the expression of inhibitors of osteoclast differentiation such as IL-10 and GM-CSF (Wada Y et al. PLoS One. 2012 7(4):e35069). WO 2005/000404 describes five pyrimidine compounds, including apilimod (Compound 12), as having inhibitory activity against osteoclast formation in an in vitro assay with an IC₅₀ of 15 nM.

As described infra, the present inventors have discovered that aplimod is a potent inhibitor of RANKL/RANK signaling.

### SUMMARY OF THE INVENTION

The present invention is based, in part, on the discovery that a PIKfyve inhibitor, apilimod, is a potent inhibitor of RANKL/RANK signaling.

The present disclosure provides methods and compositions related to the use of PIKfyve inhibitors for inhibiting RANKL/RANK signaling. Accordingly, the disclosure provides methods and compositions for treating diseases and disorders where inhibiting RANKL/RANK signaling has demonstrated therapeutic efficacy. In embodiments, the disclosure proves methods for treating certain cancers, such as multiple myeloma and giant cell tumor of bone (GCTB); methods for treating a cancer metastasis, including but limited to bone metastases; and methods for treating bone loss.

In embodiments, the disclosure provides a pharmaceutical composition comprising a PIKfyve inhibitor selected from apilimod, APY0201, and YM-201636, and pharmaceutically acceptable salts thereof, for use in a method for treating a bone loss associated disease or disorder in a patient in need thereof. In embodiments, the patient in need is one diagnosed with a disease or disorder selected from the group consisting of hypercalcemia of malignancy, bone metastasis of the breast, bone metastasis of the prostate, cancer treatment induced bone loss, multiple myeloma, rheumatoid arthritis, psoriastic arthritis, osteoporosis, skeletal unloading or disuse, sporadic Paget's disease, juvenile Paget's disease, thyrosine excess and hyperthyroidism, periprothetic bone loss, periodontal disease, and cancer metastasis. In embodiments, the PIKfyve inhibitor is apilimod free base or apilimod dimesylate. In embodiments, the PIKfyve inhibitor is apilimod dimesylate, and the amount of apilimod dimesylate in the composition is from about 0.001 mg/kg to about 1000 mg/kg.

In embodiments, the pharmaceutical composition for treating a bone loss associated disease or disorder further comprises or is administered in a combination therapy regimen with an anti-resorptive agent or anti-RANKL agent, or a combination thereof. In embodiments, the anti-resorptive agent is selected from the group consisting of progestins, polyphosphates, bisphosphonate(s), estrogen agonists, estrogen antagonists, estrogen, estrogen derivatives, and combinations thereof.

The disclosure also provides methods of treating a bone loss associated disease or disorder in a patient in need thereof, the method comprising administering to the patient a pharmaceutical composition comprising a PIKfyve inhibitor selected from apilimod, APY0201, and YM-201636, and pharmaceutically acceptable salts thereof, in accordance with any of the preceding embodiments.

The disclosure also provides a pharmaceutical composition comprising at least one PIKfyve inhibitor selected from apilimod, APY0201, and YM-201636, and pharmaceutically acceptable salts thereof, for use in treating a metastasis of a primary cancer in a patient in need thereof. In embodiments, the patient in need is a patient diagnosed with a metastatic cancer wherein the primary cancer is selected from lymphoma, multiple myeloma, breast cancer and prostate cancer. In embodiments, the metastasis is a bone metastasis. In embodiments, the primary cancer is multiple myeloma and the metastasis is a bone metastasis. In embodiments, the metastasis is refractory to standard first line therapy. In embodiments, the PIKfyve inhibitor is selected from apilimod free base and apilimod dimesylate. In embodiments, the pharmaceutical composition comprises apilimod dimesylate, the patient in need is a patient diagnosed with multiple myeloma, and the metastasis is a bone metastasis. In embodiments, the amount of apilimod dimesylate in the composition is from about 0.001 mg/kg to about 1000 mg/kg.

In embodiments, the pharmaceutical composition for treating a metastasis of a primary cancer further comprises or is administered in a combination therapy regimen with at least one additional therapeutically active agent. In embodiments, the at least one additional therapeutically active agent is selected from the group consisting of an anti-CTLA4 antibody, an anti-PD-1 agent, an anti-PD-L1 agent, and an anti-PD-L2 agent. In embodiments, the at least one additional therapeutically active agent is an anti-PD-1 antibody or the anti-CTLA4 antibody, ipilimumab.

The disclosure also provides methods of treating a metastasis of a primary cancer in a patient in need thereof, the method comprising administering to the patient a pharmaceutical composition comprising a PIKfyve inhibitor selected from apilimod, APY0201, and YM-201636, and pharmaceutically acceptable salts thereof, in accordance with any of the preceding embodiments.

The disclosure also provides a pharmaceutical composition comprising a PIKfyve inhibitor selected from apilimod, APY0201, and YM-201636, and pharmaceutically acceptable salts thereof, for use in treating giant cell tumor of bone (GCTB) in a patient in need thereof. In embodiments, the PIKfyve inhibitor is apilimod dimesylate. In embodiments, the amount of the apilimod dimesylate is from about 0.001 mg/kg to about 1000 mg/kg.

In embodiments, the pharmaceutical composition for treating GCTB further comprises or is administered in a combination therapy regimen with at least one additional therapeutically active agent. In embodiments, the at least one additional therapeutically active agent is selected from the group consisting of an anti-RANKL agent, an anti-CTLA4 antibody, an anti-PD-1 agent, an anti-PD-L1 agent, and an anti-PD-L2 agent, and combinations thereof. In embodiments, the at least one additional therapeutically active agent is selected from an anti-PD-1 antibody, the anti-CTLA4 antibody, ipilimumab, and the anti-RANKL agent, denosumab.

The disclosure also provides methods of treating GCTB in a patient in need thereof, the method comprising administering to the patient a pharmaceutical composition comprising a PIKfyve inhibitor selected from apilimod, APY0201, and YM-201636, and pharmaceutically acceptable salts thereof, in accordance with any of the preceding embodiments.

The disclosure also provides a pharmaceutical composition comprising at least one PIKfyve inhibitor selected from apilimod, APY0201, and YM-201636, and pharmaceutically acceptable salts thereof, for use in treating multiple myeloma in a patient in need thereof. In embodiments, the at least one PIKfyve inhibitor is apilimod dimesylate.

The disclosure also provides methods of treating multiple myeloma in a patient in need thereof, the method comprising administering to the patient a pharmaceutical composition comprising a PIKfyve inhibitor selected from apilimod, APY0201, and YM-201636, and pharmaceutically acceptable salts thereof, in accordance with any of the preceding embodiments.

The disclosure also provides a pharmaceutical pack or kit comprising, in separate containers or in a single container, a unit dose of at least one PIKfyve inhibitor selected from the group consisting of apilimod, APY0201, and YM-201636, and pharmaceutically acceptable salts thereof, and a unit dose of at least one additional agent. In embodiments, the at least one additional agent comprises an anti-resorptive agent or anti-RANKL agent, or a combination thereof. In embodiments, the anti-resorptive agent is selected from the group consisting of progestins, polyphosphonates, bisphosphonate(s), estrogen agonists, estrogen antagonists, estrogen, estrogen derivatives and combinations thereof.

In embodiments, the disclosure provides a method of treating a cancer or a cancer metastasis by administering a PIKfyve inhibitor in amounts sufficient to inhibit RANKL/RANK signaling in the cells of the cancer. In embodiments, the disclosure provides a method of inhibiting the progression of a cancer by administering a PIKfyve inhibitor in amounts sufficient to inhibit RANKL/RANK signaling in the cells of the cancer. In embodiments, the cells of the cancer are stromal cells or giant cells and the cancer is GCTB. In embodiments, the cells of the cancer are myeloma cells. In embodiments, the disclosure provides a method of treating, preventing or reducing the incidence of a cancer metastasis by administering a PIKfyve inhibitor in amounts sufficient to inhibit RANKL/RANK signaling in the cells of the cancer. In embodiments, the disclosure provides a method of treating a cancer metastasis by administering a PIKfyve inhibitor in amounts sufficient to inhibit RANKL/RANK signaling in the cells of the cancer. In embodiments, the cancer metastasis is a bone metastasis. In embodiments, the cancer is selected from the group consisting of breast cancer, prostate cancer, renal cancer, liver cancer, lung cancer, and skin cancer. In embodiments, the cancer metastasis is a bone metastasis and the primary cancer is selected from multiple myeloma, breast cancer, and prostate cancer.

In embodiments, the disclosure provides methods for treating bone loss in a subject in need thereof, the methods comprising administering to the subject a composition comprising an amount of at least one PIKfyve inhibitor. In embodiments, the bone loss is associated with at least one condition selected from an osteopenic disorder, an inflammatory condition, an autoimmune condition, and cancer.

In embodiments, the bone loss is associated with cancer. In embodiments, the bone loss is associated with at least one of hypercalcemia of malignancy (HCM), osteolytic bone lesions of multiple myeloma, and osteolytic bone metastases of a metastatic cancer. In embodiments, the metastatic cancer is selected from breast cancer, prostate cancer, thyroid cancer, kidney cancer, lung cancer, esophageal cancer, rectal cancer, bladder cancer, cervical cancer, ovarian cancer, and liver cancer, and gastrointestinal tract cancer. In embodiments, the metastatic cancer is breast cancer.

In embodiments, the bone loss is associated with a non-malignant bone disorder. In embodiments, the non-malignant bone disorder is selected from the group consisting of osteoporosis, Paget's disease of bone, osteogenesis imperfecta, fibrous dysplasia, primary hyperparathyroidism, familial expansile osteolysis, and expansile skeletal hyperphosphatasia. In embodiments, the bone loss is associated with a condition selected from the group consisting of familial expansile osteolysis, early-onset familial Paget's disease of bone, and expansile skeletal hyperphosphatasia.

In embodiments, the bone loss is associated psoriastic arthritis or rheumatoid arthritis. In embodiments, the bone loss is associated with periodontal disease.

In embodiments, the bone loss disease is osteoporosis. In embodiments, the osteoporosis is a primary form of osteoporosis in childhood selected from the group consisting of osteogenesis imperfecta, X-linked hypophoshatemic rickets, homocystinuria, hypophosphatasia, Wilson's disease, Menkes' kinky hair syndrome, osteoporosis-pseudoglioma syndrome, idiopathic juvenile osteoporosis, juvenile Paget's disease, early-onset Paget's disease, Ehler-Danlos syndrome, Bruck syndrome, Marfan syndrome, hypophosphatemic nephrolithiasis/osteoporosis, Hajdu-Cheney syndrome, Torg-Winchester syndrome, Shwachman-Diamond syndrome, Singleton-Merten syndrome, cleidocranial dysostosis, Stuve-Wiedemann syndrome, Cole-Carpenter syndrome, geroderma osteodysplasticum, Noonan syndrome, neonatal hyperparathyroidism, and hypocalcemic rickets. In embodiments, the disease or disorder is osteogenesis imperfecta.

In embodiments, the bone loss is associated with at least one of hypercalcemia of malignancy, bone metastasis of the breast, bone metastasis of the prostate, cancer treatment induced bone loss, multiple myeloma, rheumatoid arthritis, psoriastic arthritis, osteoporosis, skeletal unloading or disuse, sporadic Paget's disease, juvenile Paget's disease, thyrosine excess and hyperthyroidism, periprothetic bone loss, periodontal disease, and cancer metastasis.

In embodiments, the disclosure provides methods for treating multiple myeloma growth in bone in a subject in need thereof, the methods comprising administering to the subject a composition comprising an amount of at least one PIKfyve inhibitor. In embodiments, the bone growth is associated with a condition selected from an osteopenic disorder, an inflammatory condition, an autoimmune condition, and cancer.

In accordance with any of the foregoing embodiments, the PIKfyve inhibitor is selected from the group consisting of apilimod free base, apilimod dimesylate, APY0201, and YM-201636. In embodiments, the PIKfyve inhibitor is apilimod dimesylate. In embodiments, the PIKfyve inhibitor is selected from apilimod free base or pharmaceutically acceptable salt, solvate, clathrate, hydrate, polymorph, prodrug, analog or derivative thereof. In embodiments, the PIKfyve inhibitor is an active metabolite of an apilimod.

In accordance with any of the foregoing embodiments, the subject is preferably a human subject.

In accordance with any of the foregoing embodiments, the at least one PIKfyve inhibitor can be administered by any suitable route and either in the same dosage form or in a different dosage form from the optional additional agent. In embodiments, administration is via an oral, intravenous, or subcutaneous route. In embodiments, administration is once daily, twice daily, or continuous for a period of time, for example one or several days or one or several weeks. Continuous administration may be performed, for example, by using slow release dosage form that is e.g., implanted in the subject, or via continuous infusion, for example using a pump device, which also may be implanted.

In accordance with any of the foregoing embodiments, the PIKfyve inhibitor may be administered orally, for example in the form of a tablet, capsule, sublingual dosage form, or oral spray. In embodiments, the PIKfyve inhibitor is administered by injection or by addition to sterile infusion fluids for intravenous infusion and is in the form of a suitable sterile aqueous solution or dispersion, or in the form of a powder suitable for reconstitution into such a solution or dispersion.

In accordance with any of the foregoing embodiments, the PIKfyve inhibitor is apilimod, preferably apilimod dimesylate, and the amount of apilimod administered in humans is from about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents.

In accordance with any of the foregoing embodiments, the PIKfyve inhibitor, preferably apilimod, and most preferably apilimod dimesylate, is administered at a dosage regimen of 30-1000 mg/day (e.g., 30, 35, 40, 45, 50, 55, 60, 65, 70, 75, 80, 85, 90, 95, 100, 125, 150, 175, 200, 225, 250, 275, or 300 mg/day) for at least 1 week (e.g., 1, 2, 3, 4, 5, 6, 7, 8, 9, 10, 11, 12, 36, 48, or more weeks). Preferably, the compound is administered at a dosage regimen of 100-1000 mg/day for 4 or 16 weeks. Alternatively or subsequently, the compound is administered at a dosage regimen of 100 mg-300 mg twice a day for 8 weeks, or optionally, for 52 weeks. Alternatively or subsequently, the compound is administered at a dosage regimen of 50 mg-1000 mg twice a day for 8 weeks, or optionally, for 52 weeks.

In accordance with any of the foregoing embodiments, the PIKfyve inhibitor, preferably apilimod, and most preferably apilimod dimesylate, can be administered once daily, from two to five times daily, up to two times or up to three times daily, or up to eight times daily. In one embodiment, compound is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In accordance with any of the foregoing embodiments, the at least one PIKfyve inhibitor, preferably apilimod, and most preferably apilimod dimesylate, may further be combined with at least one additional active agent in a combination therapy for the treatment of a cancer, a cancer metastasis, or bone loss. In various embodiments, depending on the therapeutic regimen of the active agents, the PIKfyve inhibitor may be present in the same dosage form as the at least one additional active agent, or in a different dosage form. In embodiments, the at least one PIKfyve inhibitor is administered in a therapeutic regimen with at least one additional active agent, in the same or different dosage forms.

In embodiments of the methods for treating bone loss, the at least one additional agent is an anti-resorptive agent, an anti-RANKL agent, or a cathepsin K inhibitor, and combinations thereof. In embodiments, the anti-resorptive agent is selected from the group consisting of, a progestin, a polyphosphonate, a bisphosphonate, an estrogen receptor modulator, estrogen, an estrogen/progestin combination, an estrogen derivatives, and combinations thereof. In embodiments, the anti-RANKL agent is denosumab (Prolial™ or Xgeva™). In embodiments, the bisphosphonate is selected from the group consisting of alendronate (Fosamax™, Fosamax™ Plus D), risedronate (Actonel™, Actonel™ with Calcium), ibandronate (Boniva™), and zoledronic acid (Reclast™). In embodiments, the estrogen receptor modulator is raloxifene (Evista™). In embodiments, the anti-resorptive agent is teriparatide (Forteo™). In embodiments, the cathepsin K inhibitor is Odanacatib™.

In embodiments of the methods for treating cancer or a cancer metastasis, the at least one additional agent is selected from the group consisting of an alkylating agent, an intercalating agent, a tubulin binding agent, a corticosteroid, and combinations thereof. In embodiments, the additional therapeutic agent is selected from the group consisting of an anti-CTLA4 antibody, an anti-PD-1 agent, an anti-PD-L1 agent, and an anti-PD-L2 agent. In embodiments, the anti-CTLA4 antibody is ipilimumab. In embodiments, the additional therapeutic agent is denosumab (Prolial™ or Xgeva™). In embodiments, the cancer is GCTB and the additional therapeutic agent is denosumab.

The invention also provides a pharmaceutical pack or kit comprising, in separate containers or in a single container, a unit dose of at least one PIKfyve inhibitor, and optionally at least one additional agent, as described herein. In embodiments, the pharmaceutical pack or kit comprises at least one PIKfyve inhibitor selected from apilimod free base, apilimod dimesylate, or a racemically pure enantiomer of an active metabolite of apilimod, and combinations thereof.

### BRIEF DESCRIPTION OF THE DRAWINGS

FIGS. 1A-1D: CRISPR-induced loss of chloride voltage-gated channel 7 (CLCN7) (A), osteoporosis associated transmembrane protein 1 (OSTM1) (B), sorting nexin 10 (SNX10) (C), and lysosomal regulator transcription factor EB (TFEB) (D) confer resistance to apilimod in WSU-DLCL2B cell lymphoma. Pools cells containing guide RNAs targeted against either the indicated genes or non-targeting (NT) guide RNAs were treated with apilimod for 3 days and viability was assayed by CellTiter Glo (Promega).

FIG. 2: Apilimod treatment inhibits Cathepsin K maturation in RAW 264.7-derived osteoclasts. RAW264.7 macrophages were differentiated with 30 ng/ml RANKL for 4 days and subsequently treated with RANKL and the indicated concentration of apilimod for 24 hours prior to harvesting lysates and performing western blot for the indicated protein.

FIG. 3: Apilimod treatment inhibits RANKL-induced differentiation of tartrate-resistant acid phosphatase (TRAP) positive, multinucleated osteoclasts from RAW264.7 macrophages. RAW264.7 macrophages were differentiated with either vehicle or 30 ng/mL RANKL for a total of 5 days. For the last 3 days of differentiation, cells were co-treated with the indicated concentration of apilimod. Cells were subsequently stained for TRAP. Arrows highlight giant TRAP positive, multinucleated osteoclasts.

FIGS. 4A-4B: Inhibition of RANKL-induced differentiation of RAW264.7-derived osteoclasts by apilimod as indicated by a reduction in the number of TRAP-positive multinucleated cells (A) or giant osteoclasts (B). The average of triplicate wells was determined and percentages relative to untreated are shown.

FIG. 5A-5B: Effect of apilimod treatment on the RNA expression of osteogenic factors RANK, c-Fos, microphthalmia-associated transcription factor (MITF), PU.1, TNF receptor associated factor 6 (TRAF6) and osteoprotegerin (OPG) in undifferentiated (A) or differentiated (B) RAW264.7 macrophages as assessed by quantitative PCR.

FIGS. 6A-6B: Graphical representation of the effect of apilimod on periodontal bone resorption (A). Daily oral doses of apilimod (8 and 20 mg/kg) reduce bone loss (B).

FIG. 7: Positron emission tomography-computer tomography (PET-CT) scan of a patient with diffuse large B cell lymphoma (DLBCL). Left image was taken on day 2 as a baseline; Right image was taken two weeks after end of treatment (100 mg apilimod dimesylate BID for 6 weeks).

FIG. 8: Effect of apilimod on hind limb paralysis in the MPC-11 syngeneic model.

FIG. 9: Effect of apilimod on bone marrow architecture in the MPC-11 synergeic model.

### DETAILED DESCRIPTION OF THE INVENTION

The present disclosure provides compositions and methods related to the use of PIKfyve inhibitors for inhibiting cellular RANKL/RANK signaling. Accordingly, the disclosure provides methods relating to the treatment, and in some embodiments, prophylaxis, of certain diseases and disorders whose clinical pathology is characterized by inappropriate or excessive RANKL/RANK signaling. Thus, the disclosure provides, in various embodiments, methods for treating a cancer, a cancer metastases, and bone loss.

In embodiments, the invention provides compositions and methods for the treatment of cancer, cancer metastases, and bone loss in a subject by administering to the subject an amount of at least one PIKfyve inhibitor, preferably apilimod, and most preferably apilimod dimesylate. In embodiments, the amount is effective to inhibit RANKL/RANK signaling in target cells of the subject. In embodiments, the amount is a therapeutically effective amount. In embodiments, the at least one PIKFyve inhibitor is selected from the group consisting of apilimod, APY0201, and YM201636, or a pharmaceutically acceptable salt, solvate, clathrate, hydrate, polymorph, metabolite, prodrug, analog or derivative thereof. In embodiments, the at least one PIKfyve inhibitor is apilimod, preferably apilimod dimesylate.

In embodiments, the disclosure provides methods of inhibiting bone loss. In embodiments, the bone loss is associated with a disease, disorder, or condition in the subject. Examples of such disorders include, without limitation, periodontal disease, non-malignant bone disorders, including (e.g., osteoporosis, Paget's disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (e.g., hypercalcemia of malignancy (HCM), osteolytic bone lesions of multiple myeloma and osteolytic bone metastases of breast cancer and other metastatic cancers). In embodiments, the disease or disorder is an autoinflammatory bone disorder, for example chronic non-bacterialosteomyelitis (CNO), synovitis, acne, pustulosis, hyperostosis, osteitis syndrome, Majeed syndrome, deficiency of interleukin-1 receptor antagonist (DIRA), and cherubism.

In embodiments, the bone loss is associated with at least one of multiple myeloma, a metastatic solid tumor, osteoporosis, rheumatoid arthritis, periodontal disease, Paget's disease of bone, familial expansile osteolysis, and expansile skeletal hyperphosphatasia.

In embodiments, the bone loss is associated with osteoporosis. In embodiments, the osteoporosis is a primary form of osteoporosis in childhood selected from the group consisting of osteogenesis imperfecta, X-linked hypophoshatemic rickets, homocystinuria, hypophosphatasia, Wilson's disease, Menkes' kinky hair syndrome, osteoporosis-pseudoglioma syndrome, idiopathic juvenile osteoporosis, juvenile Paget's disease, early-onset Paget's disease, Ehler-Danlos syndrome, Bruck syndrome, Marfan syndrome, hypophosphatemic nephrolithiasis/osteoporosis, Hajdu-Cheney syndrome, Torg-Winchester syndrome, Shwachman-Diamond syndrome, Singleton-Merten syndrome, cleidocranial dysostosis, Stuve-Wiedemann syndrome, Cole-Carpenter syndrome, geroderma osteodysplasticum, Noonan syndrome, neonatal hyperparathyroidism, and hypocalcemic rickets. In embodiments, the primary form of osteoporosis in childhood is osteogenesis imperfecta.

In a embodiments, the bone loss is associated with at least one of hypercalcemia of malignancy, bone metastasis of the breast, bone metastasis of the prostate, cancer treatment induced bone loss, multiple myeloma, rheumatoid arthritis, psoriastic arthritis, osteoporosis, skeletal unloading or disuse, sporadic Paget's disease, juvenile Paget's disease, thyrosine excess and hyperthyroidism, periprothetic bone loss, periodontal disease, and cancer metastasis.

In embodiments, the invention provides methods for treating or preventing cancer or a cancer metastasis in a subject by administering to the subject a therapeutically effective amount of at least one PIKfyve inhibitor, preferably apilimod, and most preferably apilimod dimesylate. In embodiments, the cancer is selected from the group consisting of multiple myeloma, breast cancer, prostate cancer, renal cancer, liver cancer, lung cancer, and skin cancer. In embodiments, the cancer is multiple myeloma, breast or prostate cancer. In embodiments, the cancer is GCTB. In embodiments, the amount is effective to inhibit cellular PIKfyve activity in the cells of the cancer and/or inhibit the expression of RANK on CD4+ and CD8+ T-cells, and/or inhibit RANKL/RANK signaling in the cells of the cancer.

In accordance with any of the embodiments described here, the at least one PIKfyve inhibitor is apilimod, preferably apilimod dimesylate. Apilimod is a selective inhibitor of PIKfyve (Cai et al. 2013 Chem. & Biol. 20:912-921). Based upon its ability to inhibit IL-12/23 production, apilimod has been suggested as useful for treating inflammatory and autoimmune diseases such as rheumatoid arthritis, sepsis, Crohn's disease, multiple sclerosis, psoriasis, or insulin dependent diabetes mellitus, and in cancers where these cytokines were believed to play a pro-proliferative role.

In embodiments of the methods and compositions described here, the apilimod may be apilimod free base or a pharmaceutically acceptable salt, solvate, clathrate, hydrate, polymorph, prodrug, analog or derivative thereof, as described below. The structure of apilimod is shown in Formula I:

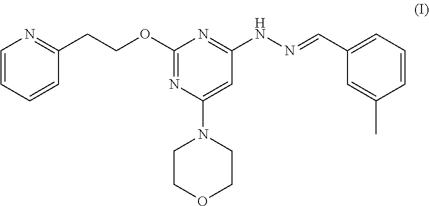 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0.

Apilimod can be prepared, for example, according to the methods described in U.S. Pat. Nos. 7,923,557, and 7,863,270, and WO 2006/128129.

In embodiments, the apilimod for use in the compositions and methods of the invention is the free base or dimesylate salt form, MW 610.7 (dimesylate salt); tPSA 83.1; pKa 5.39 (±0.03), 4.54 (±0.27); HBD 1. The apilimod dimesylate salt is highly water soluble (>25 mg/mL) and shows moderate permeability (>70% in rats). In embodiments, an active metabolite of apilimod may be used. Six primary metabolites were identified in rat and human microsomal and hepatocyte stability studies. Human, rat, rabbit and dog studies showed a qualitatively similar metabolic profile. Tₘₐₓ generally occurred within 1 or 2 hours after the oral dose, consistent with the rapid elimination of this compound from the circulation. Reaction phenotyping studies indicated that CYP3A4 and to a lesser extent CYP1A2 and/or CYP2D6, contribute to metabolism. The primary metabolites are short-lived in circulation. Both apilimod free base and the dimesylate salt are highly bound (>990%) to rat, dog and human plasma proteins.

In embodiments, the at least one PIKfyve inhibitor is selected from APY0201 and YM-201636.

The chemical name of APY0201 is (E)-4-(5-(2-(3-methylbenzylidine)hydrazinlyl)-2-(pyridine-4-yl)pyrazolol[1,5-a]pyrimidin-7-yl)morpholine. APY0201 is a selective PIKfyve inhibitor (Hayakawa et al. 2014 Bioorg. Med. Chem. 22:3021-29). APY0201 directly interacts with the ATP-binding site of PIKfyve kinase, which leads to suppression of PI(3,5)P₂ synthesis, which in turn suppresses the production of IL-12/23.

The chemical name for YM201636 is 6-amino-N-(3-(4-morpholinopyrido[3′,2′: 4,5]furo[3,2-d]pyrimidin-2-yl)phenyl)nicotinamide (CAS number is 371942-69-7). YM201636 is a selective inhibitor of PIKfyve (Jefferies et al. EMBO rep. 2008 9:164-170). It reversibly impairs endosomal trafficking in NIH3T3 cells, mimicking the effect produced by depleting PIKfyve with siRNA. YM201636 also blocks retroviral exit by budding from cells, apparently by interfering with the endosomal sorting complex required for transport (ESCRT) machinery. In adipocytes, YM-201636 also inhibits basal and insulin-activated 2-deoxyglucose uptake (IC₅₀=54 nM).

As used herein, the term “pharmaceutically acceptable salt,” is a salt formed from, for example, an acid and a basic group of a compound. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salts. In a preferred embodiment, the salt of apilimod comprises methanesulfonate.

The term “pharmaceutically acceptable salt” also refers to a salt prepared from a compound having an acidic functional group, such as a carboxylic acid functional group, and a pharmaceutically acceptable inorganic or organic base.

The term “pharmaceutically acceptable salt” also refers to a salt prepared from a compound having a basic functional group, such as an amino functional group, and a pharmaceutically acceptable inorganic or organic acid.

The salts of the compounds described herein can be synthesized from the parent compound by conventional chemical methods such as methods described in Pharmaceutical Salts: Properties, Selection, and Use, P. Hemrich Stalil (Editor), Camille G. Wermuth (Editor), ISBN: 3-90639-026-8, August 2002. Generally, such salts can be prepared by reacting the parent compound with the appropriate acid in water or in an organic solvent, or in a mixture of the two.

One salt form of a compound described herein can be converted to the free base and optionally to another salt form by methods well known to the skilled person. For example, the free base can be formed by passing the salt solution through a column containing an amine stationary phase (e.g. a Strata-NH₂ column). Alternatively, a solution of the salt in water can be treated with sodium bicarbonate to decompose the salt and precipitate out the free base. The free base may then be combined with another acid using routine methods.

As used herein, the term “polymorph” means a solid crystalline form of a compound of the present invention. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present invention or a salt thereof, which further includes a stoichiometric or non-stoichiometric amount of water bound by non-covalent intermolecular forces.

As used herein, the term “clathrate” means a compound of the present invention or a salt thereof in the form of a crystal lattice that contains spaces (e.g., channels) that have a guest molecule (e.g., a solvent or water) trapped within.

As used herein, the term “prodrug” means a derivative of a compound described herein that can hydrolyze, oxidize, or otherwise react under biological conditions (in vitro or in vivo) to provide a compound of the invention. Prodrugs may only become active upon such reaction under biological conditions, or they may have activity in their unreacted forms. Examples of prodrugs contemplated in this invention include, but are not limited to, analogs or derivatives of a compound described herein that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by Burger's Medicinal Chemistry and Drug Discovery (1995) 172-178, 949-982 (Manfred E. Wolff ed., 5th ed).

In addition, some of the compounds suitable for use in the methods of in this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms. All such isomeric forms of these compounds are expressly included in the present invention. The compounds of this invention can also be represented in multiple tautomeric forms, in such instances, the invention expressly includes all tautomeric forms of the compounds described herein (e.g., there may be a rapid equilibrium of multiple structural forms of a compound), the invention expressly includes all such reaction products). All such isomeric forms of such compounds are expressly included in the present invention. All crystal forms of the compounds described herein are expressly included in the present invention.

As used herein, the term “solvate” or “pharmaceutically acceptable solvate,” is a solvate formed from the association of one or more solvent molecules to one of the compounds disclosed herein. The term solvate includes hydrates (e.g., hemi-hydrate, mono-hydrate, dihydrate, trihydrate, tetrahydrate, and the like).

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. As used herein, the term “derivative” refers to compounds that have a common core structure, and are substituted with various groups as described herein.

### Methods of Treatment

The disclosure provides methods for inhibiting RANKL/RANK signaling using PIKfyve inhibitors and related compositions and methods. The methods relate generally to treating diseases and disorders where RANKL/RANK signaling is implicated in clinical pathology.

In embodiments, the disclosure provides methods for the treatment of bone loss in a subject in need thereof by administering to the subject an amount of at least one PIKfyve inhibitor.

In embodiments, the disclosure provides methods for treating cancer or a cancer metastasis in a subject in need thereof, the methods comprising administering to the subject an amount of at least one PIKfyve inhibitor. In embodiments of the methods for treating a cancer metastasis, the cancer is selected from the group consisting of multiple myeloma, breast cancer, prostate cancer, renal cancer, liver cancer, lung cancer, and skin cancer. In embodiments, the cancer is multiple myeloma, breast cancer or prostate cancer.

In embodiments, the disclosure provides methods for treating cancer where the cancer is giant cell tumor of bone (GCTB) in a subject in need thereof, the methods comprising administering to the subject an amount of at least one PIKfyve inhibitor.

In accordance with the methods described here, the amount is an amount effective to inhibit RANKL/RANK signaling in target cells of the bone tissue or cancer of the subject. In embodiments, the target cells are selected from T cells, osteoclasts and cells of a cancer, including stromal cells and giant cells in the case of GCTB. In embodiments, the cells of the cancer are stromal cells or giant cells and the cancer is giant cell tumor of bone (GCTB).

In embodiments, the amount is an amount effective to achieve one or more of the following: inhibit cellular PIKfyve activity, inhibit cathepsin K processing in osteoclasts, inhibit RANKL-stimulated osteoclastogenesis, inhibit the expression of RANK on CD4+ and CD8+ T-cells. In embodiments, the amount is an amount effective to block the differentiation of osteoclast precursors. In embodiments, the amount is an amount sufficient to reduce bone loss (alternatively, “bone mass”). In embodiments, the amount is an amount effective to block the resorptive activity of mature osteoclasts. In embodiments, the amount is an amount sufficient to reduce net bone loss. In embodiments, the amount is an amount effective to suppress the rate of bone resorption.

In embodiments, the amount is an amount sufficient to slow the progression of a cancer in the subject, by reducing the incidence of new metastases and/or by decreasing the number and/or size of metastatic lesions in the subject. In embodiments, treating a cancer metastasis according to the methods described herein results in a decrease in the number and/or size of metastatic lesions in tissue or organs distant from the primary tumor site. In embodiments, the tissue is bone tissue. In embodiments, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%.

In accordance with any of the embodiments described here, the at least one PIKfyve inhibitor is selected from apilimod, APY0201, YM-201636 or a pharmaceutically acceptable salt, solvate, clathrate, hydrate, polymorph, metabolite, prodrug, analog or derivative thereof. In embodiments, the PIKfyve inhibitor is apilimod dimesylate. In embodiments, the PIKfyve inhibitor is selected from apilimod free base or pharmaceutically acceptable salt, solvate, clathrate, hydrate, polymorph, prodrug, analog or derivative thereof. In embodiments, the PIKfyve inhibitor is apilimod, an active metabolite of apilimod, or a combination thereof.

The disclosure further provides the use of at least one PIKfyve inhibitor for the preparation of a medicament useful for the treatment of bone loss diseases and cancer or a cancer metastasis, as described herein. In embodiments, the cancer is multiple myeloma or GCTB.

In embodiments, the effective amount of the PIKfyve inhibitor, preferably apilimod, and most preferably apilimod dimesylate is from about 0.001 mg/kg to about 1000 mg/kg, more preferably 0.01 mg/kg to about 100 mg/kg, more preferably 0.1 mg/kg to about 10 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents. See e.g., U.S. Pat. No. 7,863,270, incorporated herein by reference.

In embodiments, the therapeutically effective amount of the PIKfyve inhibitor, preferably apilimod, and most preferably apilimod dimesylate, in humans is administered at a dosage regimen of about 30-1000 mg/day (e.g., 30, 35, 40, 45, 50, 55, 60, 65, 70, 75, 80, 85, 90, 95, 100, 125, 150, 175, 200, 225, 250, 275, or 300 mg/day) for at least 1 week (e.g., 1, 2, 3, 4, 5, 6, 7, 8, 9, 10, 11, 12, 36, 48, or more weeks). Preferably, the compound is administered at a dosage regimen of 100-1000 mg/day for 4 or 16 weeks. Alternatively or subsequently, the compound is administered at a dosage regimen of 100 mg-300 mg twice a day for 8 weeks, or optionally, for 52 weeks. Alternatively or subsequently, the compound is administered at a dosage regimen of 50 mg-1000 mg twice a day for 8 weeks, or optionally, for 52 weeks.

In embodiments, the at least one PIKfyve inhibitor, preferably apilimod, and most preferably apilimod dimesylate, is administered once daily, from two to five times daily, up to two times or up to three times daily, or up to eight times daily. In embodiments, the compound is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

A “subject” includes a mammal. The mammal can be e.g., any mammal, e.g., a human, primate, vertebrate, bird, mouse, rat, fowl, dog, cat, cow, horse, goat, camel, sheep or a pig. Preferably, the subject is a human. The term “patient” refers to a human subject, preferably a human subject diagnosed with a disease or disorder.

As used herein, “treatment”, “treating” or “treat”” refer to the reduction of the severity, duration, or progression of the disease or disorder being treated and may include the amelioration of one or more symptoms or complications associated with the disease or disorder.

### Combination Therapies

The disclosure also provides methods comprising combination therapy. As used herein, “combination therapy” or “co-therapy” includes the administration of a therapeutically effective amount of a PIKfyve inhibitor, preferably apilimod, and most preferably apilimod dimesylate, with at least one additional active agent, as part of a specific treatment regimen intended to provide a beneficial effect from the co-action of the active agents in the regimen. In embodiments, the additional active agent may include a therapeutic agent conventionally used to prevent or treat bone loss or diseases or conditions associated with bone loss. In embodiments, the additional active agent may include a therapeutic agent conventionally used to prevent or treat cancer metastases. “Combination therapy” is not intended to encompass the administration of two or more therapeutic agents as part of separate monotherapy regimens that incidentally and arbitrarily result in a beneficial effect that was not intended or predicted.

In embodiments, the disclosure provides methods of treating a subject for bone loss using a combination therapy comprising a PIKfyve inhibitor, preferably apilimod, and most preferably apilimod dimesylate, and at least one additional therapeutic or non-therapeutic agent, or both. In embodiments, the additional therapeutic agent is selected from an anti-resorptive agent, including, for example, progestins, polyphosphonates, bisphosphonate(s), estrogen agonists/antagonists, estrogen, estrogen/progestin combinations, and estrogen derivatives. Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, dthynodiol diacetate, etonogestrel, flurogestone acetate, gestaclone, gestodene, gestonorone caproate, gestrinone, haloprogesterone, hydroxyprogesterone, caproate, levonorgestrel, lynestrenol, medrogestone, medroxyprogesterone acetate, melengestrol acetate, methynodiol diacetate, norethindrone, norethindrone acetate, norethynodrel, norgestimate, n & r gestomet, norgestrel, oxogestone phenpropionate, progesterone, quingestanol acetate, quingestrone, and tigestol. Preferred progestins are medroxyprogestrone, norethindrone and norethynodrel.

In embodiments, the anti-resorptive agent is selected from the group consisting of, progestins, polyphosphonates, bisphosphonate(s), estrogen agonists/antagonists, estrogen, estrogen/progestin combinations, and estrogen derivatives and combinations thereof. In embodiments, the at least one additional agent is a bisphosphonate anti-resorptive agent selected from the group consisting of alendronate (Fosamax™, Fosamax™ Plus D), risedronate (Actonel™, Actonel™ with Calcium), ibandronate (Boniva™), and zoledronic acid (Reclast™). In embodiments, the at least one additional agent is an anti-resorptive agent selected from the group consisting of raloxifene (Evista™) and denosumab (Prolial™ or Xgeva™). In embodiments, the at least one additional agent is an anabolic agent such as teriparatide (Forteo™).

In embodiments the at least one additional therapeutic agent is a cathepsin K inhibitor. In embodiments, the cathepsin K inhibitor is Odanacatib™.

In embodiments the at least one additional therapeutic agent is an estrogen agonist/antagonist. In embodiments, the term estrogen agonist/antagonist refers to compounds which bind with the estrogen receptor, inhibit bone turnover and/or prevent bone loss. In particular, estrogen agonists may include chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue; and blocking the actions of estrogen in one or more tissues. Such activities are readily determined by those skilled in the art of standard assays including estrogen receptor binding assays, standard bone histomorphometric and densitometer methods.

In embodiments the at least one additional therapeutic agent is selected from a bisphosphonate (such as etidronate (Didronel®, Procter & Gamble), pamidronate (Aredia®, Novartis), and alendronate (Fosamax®, Merck)), tiludronate (Skelid®, Sanofi-Synthelabo, Inc.), risedronate (Actonel®, Procter & Gamble/Aventis), calcitonin (Miacalcin®), estrogens (Climara®, Estrace®, Estraderm®, Estratab®, Ogen®, Ortho-Est®, Vivelle®, Premarin®, and others) estrogens and progestins (Activella™, FemHrt®, Premphase®, Prempro®, and others), parathyroid hormone and portions thereof, such as teriparatide (Forteo®, Eli Lilly and Co.), selective estrogen receptor modulators (SERMs) (such as raloxifene (Evista®)) and treatments currently under investigation (such as other parathyroid hormones, sodium fluoride, vitamin D metabolites, and other bisphosphonates and selective estrogen receptor modulators).

In embodiments, the at least one additional therapeutic agent is selected from bone morphogenic factors designated BMP-1 through BMP-12; transforming growth factor-β (TGF-β) and TGF-β family members; interleukin-1 (IL-) inhibitors, including, but not limited to, IL-Ira and derivatives thereof and Kineret™ anakinra, TNFα inhibitors, including, but not limited to, a soluble TNFα receptor, Enbrel™, etanercept, anti-TNFα antibodies, Remicade™, infliximab, and D2E7 antibody; parathyroid hormone and analogs thereof, parathyroid related protein and analogs thereof; E series prostaglandins; bisphosphonates (such as alendronate and others); bone-enhancing minerals such as fluoride and calcium; non-steroidal anti-inflammatory drugs (NSAIDs), including COX-2 inhibitors, such as Celebrex™, celecoxib, and Vioxx™, rofecoxib, immunosuppressants, such as methotrexate or leflunomide; serine protease inhibitors such as secretory leukocyte protease inhibitor (SLPI); IL-6 inhibitors (e.g., antibodies to IL-6), IL-8 inhibitors (e.g., antibodies to IL-8); IL-18 inhibitors (e.g., IL-18 binding protein or IL-18 antibodies); Interleukin-1 converting enzyme (ICE) modulators; fibroblast growth factors FGF-1 to FGF-10 and FGF modulators; PAF antagonists; keratinocyte growth factor (KGF), KGF-related molecules, or KGF modulators; matrix metalloproteinase (MMP) modulators; Nitric oxide synthase (NOS) modulators, including modulators of inducible NOS; modulators of glucocorticoid receptor; modulators of glutamate receptor; modulators of lipopolysaccharide (LPS) levels; and noradrenaline and modulators and mimetics thereof.

In embodiments, the therapeutic agent is a steroid or a non-steroidal anti-inflammatory agent. Useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof.

In embodiments, the disclosure provides methods of treating a cancer metastasis in a subject in need thereof using a combination therapy comprising a PIKfyve inhibitor, preferably apilimod, and most preferably apilimod dimesylate, and at least one additional therapeutic or non-therapeutic agent, or both. In embodiments, the additional therapeutic agent is selected from the group consisting of an alkylating agent, an intercalating agent, a tubulin binding agent, a corticosteroid, and combinations thereof.

In embodiments, the additional therapeutic agent is selected from the group consisting of an anti-CTLA4 antibody, an anti-PD-1 agent, an anti-PD-L1 agent, and an anti-PD-L2 agent. In embodiments, the anti-CTLA4 antibody is ipilimumab.

In embodiments, the at least one additional active agent is a therapeutic agent selected from the group consisting of ibrutinib, rituximab, doxorubicin, prednisolone, vincristine, velcade, and everolimus, and combinations thereof. In embodiments, the at least one additional active agent is a therapeutic agent selected from cyclophosphamide, hydroxydaunorubicin (also referred to as doxorubicin or Adriamycin™), vincristine (also referred to as Oncovin™), prednisone, prednisolone, and combinations thereof. In embodiments, the anti-cancer agent is selected from an inhibitor of EZH2, e.g., EPZ-6438. In embodiments, the at least one additional active agent is a therapeutic agent selected from taxol, vincristine, doxorubicin, temsirolimus, carboplatin, ofatumumab, rituximab, and combinations thereof. In embodiments, the at least one additional active agent is a therapeutic agent selected from chlorambucil, ifosphamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In embodiments, the at least one additional active agent is a therapeutic agent selected from alemtuzumab, bevacizumab, catumaxomab, cetuximab, edrecolomab, gemtuzumab, ofatumumab, panitumumab, rituximab, trastuzumab, eculizumab, efalizumab, muromab-CD3, natalizumab, adalimumab, afelimomab, certolizumab pegol, golimumab, infliximab, basiliximab, canakinumab, daclizumab, mepolizumab, tocilizumab, ustekinumab, ibritumomab tiuxetan, tositumomab, abagovomab, adecatumumab, alemtuzumab, anti-CD30 monoclonal antibody Xmab2513, anti-MET monoclonal antibody MetMab, apolizumab, apomab, arcitumomab, basiliximab, bispecific antibody 2B1, blinatumomab, brentuximab vedotin, capromab pendetide, cixutumumab, claudiximab, conatumumab, dacetuzumab, denosumab, eculizumab, epratuzumab, ertumaxomab, etaracizumab, figitumumab, fresolimumab, galiximab, ganitumab, gemtuzumab ozogamicin, glembatumumab, ibritumomab, inotuzumab ozogamicin, ipilimumab, lexatumumab, lintuzumab, lintuzumab, lucatumumab, mapatumumab, matuzumab, milatuzumab, monoclonal antibody CC49, necitumumab, nimotuzumab, ofatumumab, oregovomab, pertuzumab, ramacurimab, ranibizumab, siplizumab, sonepcizumab, tanezumab, tositumomab, trastuzumab, tremelimumab, tucotuzumab celmoleukin, veltuzumab, visilizumab, volociximab, and zalutumumab.

In embodiments, the additional therapeutic agent is denosumab (Prolial™ or Xgeva™). In embodiments, the cancer is GCTB and the additional therapeutic agent is denosumab.

In embodiments, the methods include administration of at least one additional active agent that is a non-therapeutic agent, for which the beneficial effect of the combination may relate to the mitigation of toxicity, side effect, or adverse event associated with a therapeutically active agent in the combination. In embodiments, the non-therapeutic agent mitigates one or more side effects of apilimod, the one or more side effects selected from any of nausea, vomiting, headache, dizziness, lightheadedness, drowsiness and stress. In one aspect of this embodiment, the non-therapeutic agent is an antagonist of a serotonin receptor, also known as 5-hydroxytryptamine receptors or 5-HT receptors. In one aspect, the non-therapeutic agent is an antagonist of a 5-HT3 or 5-HT1a receptor. In one aspect, the non-therapeutic agent is selected from the group consisting of ondansetron, granisetron, dolasetron and palonosetron. In another aspect, the non-therapeutic agent is selected from the group consisting of pindolol and risperidone.

In the context of combination therapy, administration of the PIKfyve inhibitor, preferably apilimod, and most preferably apilimod dimesylate, may be simultaneous with or sequential to the administration of the one or more additional active agents. In embodiments, administration of the different components of a combination therapy may be at different frequencies. The one or more additional agents may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a compound of the present invention.

The one or more additional active agents can be formulated for co-administration with an apilimod composition in a single dosage form, as described in greater detail herein. The one or more additional active agents can be administered separately from the dosage form that comprises the PIKfyve inhibitor. When the additional active agent is administered separately from the PIKfyve inhibitor, it can be by the same or a different route of administration as the PIKfyve inhibitor.

Preferably, the administration of PIKfyve inhibitor in combination with one or more additional agents provides a synergistic response in the subject being treated. In this context, the term “synergistic” refers to the efficacy of the combination being more effective than the additive effects of either single therapy alone. The synergistic effect of a combination therapy according to the invention can permit the use of lower dosages and/or less frequent administration of at least one agent in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy).

In certain embodiments the at least one PIKfyve inhibitor, preferably apilimod, and most preferably apilimod dimesylate, is provided in a single dosage form in combination with one or more additional therapeutic agents. In another embodiment, apilimod is provided in combination with one or more additional PIKfyve inhibitors, for example APY0201 and YM201636. Where more than one therapeutic agent is present in a single dosage form, the therapeutically effective amount is based upon the total amount of therapeutic agents in the dosage form.

In one embodiment the at least one PIKfyve inhibitor is provided in a separate dosage form from the one or more additional therapeutic agents. Separate dosage forms are desirable, for example, in the context of a combination therapy in which the therapeutic regimen calls for administration of different therapeutic agents at different frequencies or under different conditions, or via different routes.

In one embodiment, administration of the at least one PIKfyve inhibitor as described herein is accomplished via an oral dosage form suitable for oral administration. In another embodiment administration is by an indwelling catheter, a pump, such as an osmotic minipump, or a sustained release composition that is, for example, implanted in the subject.

### Pharmaceutical Compositions and Formulations

The disclosure also provides pharmaceutical compositions comprising an amount of at least one PIKfyve inhibitor and at least one pharmaceutically acceptable excipient or carrier. Preferably, the amount is a therapeutically effective amount.

In embodiments, the PIKfyve inhibitor is selected from one or more of apilimod, APY0201, YM-201636, and pharmaceutically acceptable salts, solvates, clathrates, hydrates, polymorphs, metabolites, prodrugs, analogs and derivatives thereof. In one embodiment, the PIKfyve inhibitor is apilimod, preferably apilimod dimesylate.

In embodiments, the at least one PIKfyve inhibitor is further combined with at least one additional therapeutic agent in a single dosage form. Suitable additional therapeutic agents are described in detail supra.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating bone loss disease are described above.

A dose may be provided in unit dosage form. For example, the unit dosage form can comprise 1 nanogram to 2 milligrams, or 0.1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

The pharmaceutical compositions can take any suitable form (e.g., liquids, aerosols, solutions, inhalants, mists, sprays; or solids, powders, ointments, pastes, creams, lotions, gels, patches and the like) for administration by any desired route (e.g., pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present invention may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants the HLB values can differ by as much as about 8 HLB units, depending upon the empirical method chosen to determine the HLB value.

Among the surfactants for use in the compositions of the invention are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### EXAMPLES

The present invention is based, in part, on the surprising discovery that PIKfyve kinase is a potent inhibitor of RANKL/RANK signaling.

The present invention is further based, in part, on the surprising discovery that PIKfyve kinase activity is critical for the normal functioning of the cellular processes underlying the maintenance of bone density. This discovery was made serendipitously in a screen for genes essential for the cytotoxic effects of the PIKfyve inhibitor apilimod in lymphoma cells. Surprisingly, among the genes found to be necessary for apilimod-induced cytotoxicity were the osteopetrosis associated genes OSTM1 and CLCN7. Osteopetrosis is an extremely rare inherited disorder which causes the bones harden and becoming denser, unlike the more common osteoporosis, in which the bones become less dense and more brittle. As discussed below, further work was done to explore the effects of PIKfyve on the cellular processes of bone maintenance. This work showed that inhibiting PIKfyve blocked cathepsin K processing in osteoclasts derived from RAW264.7 macrophages and as well as RANKL-stimulated osteoclastogenesis of RAW264.7 macrophages in vitro. These effects were surprising because the only other published connection between PIKfyve and bone density showed that loss of PIKfyve resulted in decreased bone mineral density (Min S H et al. Nature Commun. 2014 5: 4691).

Further, as described in the examples below, it was discovered that apilimod potently blocked RANKL-stimulated osteoclastogenesis of RAW264.7 macrophages in vitro by blocking the expression of RANK receptor. RANKL/RANK signaling has been associated with cancer progression and metastasis in breast, prostate, and renal cancer systems (Palafox M et al. Cancer Res. 2012 72(11):2879-88; Santini D et al. PLoS One. 2011 6(4):e19234; Mikami S et al. J Pathol. 2009 218(4):530-39; Tan W et al. Nature. 2011 470(7335):548-53; Luo J L et al. Nature. 2007 446(7136):690-4). T-cell derived RANKL has been linked to the promotion of metastases in breast and prostate cancer mouse models and anti-RANKL was shown to synergize with anti-CTLA4 in blocking melanoma lung metastasis in mice (Smyth M J et al. J Clin Oncol. 2016 34(12):e104-6).

We further show here that apilimod impairs multiple myeloma growth in bone in the systemic MPC-11 syngeneic mouse model. As discussed below, apilimod treatment prevented hind limb paralysis and significantly reduced tumor burden in this animal model.

The potent anti-RANKL/RANK activity of apilimod demonstrated by these studies indicates that apilimod, and possibly other PIKfyve inhibitors, may be clinically useful for preventing pathological bone loss, for example as occurs in osteoporosis and related conditions, and as anti-cancer agents for inhibiting bone-related cancer progression and metastasis, either alone or in combination with other therapeutic agents.

### Example 1: Apilimod-Induced Alterations in Cytokine Profiles and Endolysosomal Dynamics Block Both the Differentiation of Osteoclast Precursors and Resorptive Activity of Mature Osteoclasts

The target of apilimod is the lipid kinase phosphatidylinositol-3-phosphate 5-kinase (PIKfyve), which phosphorylates endosomal PI3P to generate the phosphoinositide PI(3,5)P2 (Boyle W J et al. Nature. 2003 423(6937):337-42). Loss of PI(3,5)P2 through PIKfyve inhibition is associated with extensive endomembrane vacuolization and disruption of endolysosomal trafficking. Apilimod-induced inhibition of PIKfyve is cytotoxic to B-cell lymphoma though a lysosomal-dependent mechanism was demonstrated.

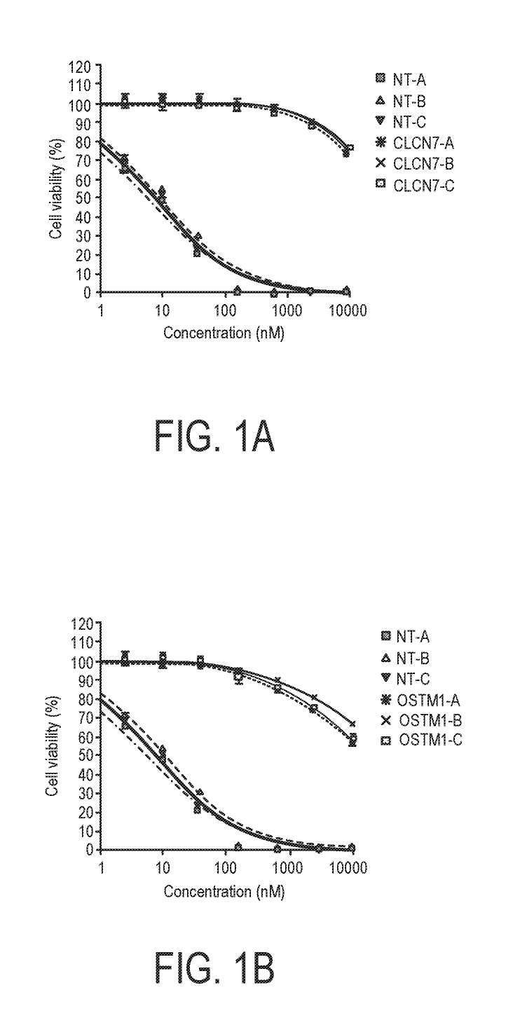

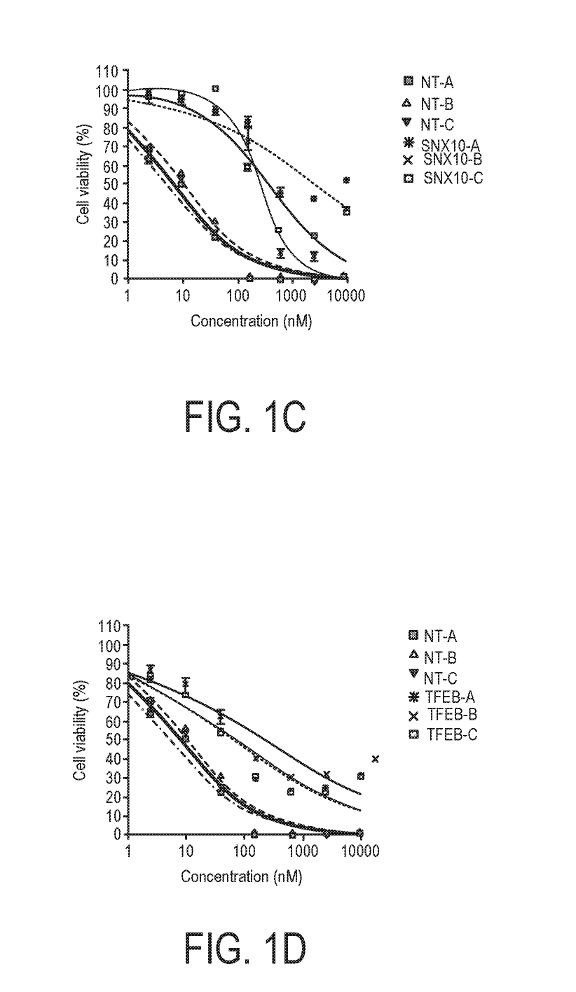

A genome-wide CRISPR screen for factors conferring resistance of B-cell lymphoma to apilimod uncovered the lysosomal regulator TFEB as well as the lysosomal and osteopetrosis-associated genes CLCN7, OSTM1, and SNX10 as mediators of apilimod-induced cytotoxicity (see FIGS. 1A-1D).

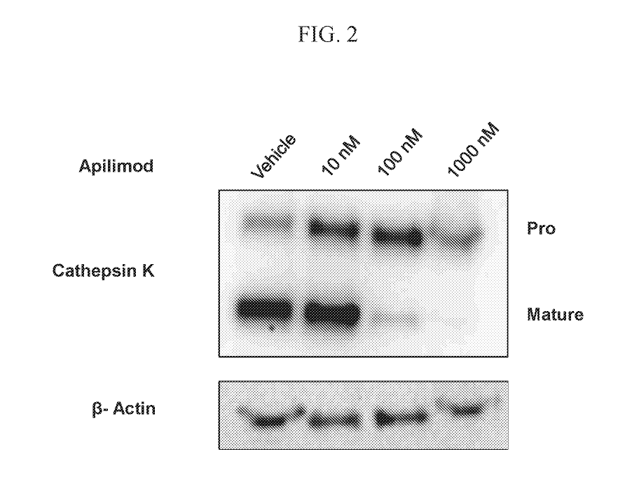

Furthermore the endolysosomal trafficking defect induced by apilimod results in the inhibition of cathepsin K processing in osteoclasts derived from RAW264.7 macrophages (see FIG. 2).

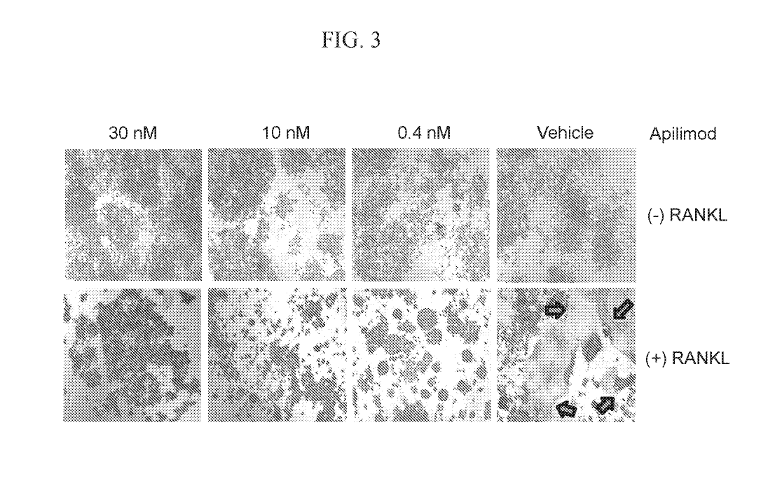

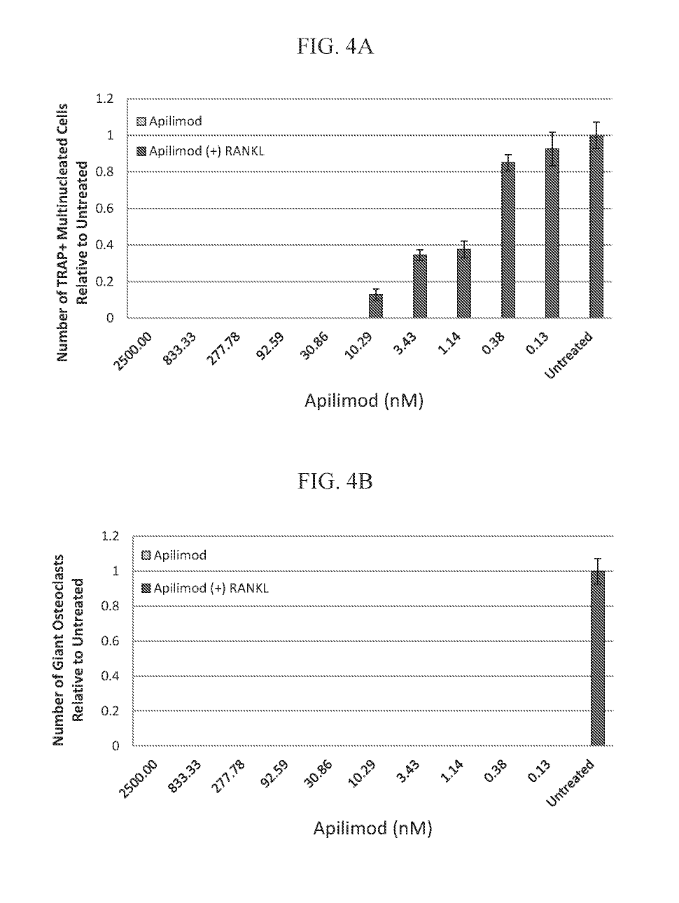

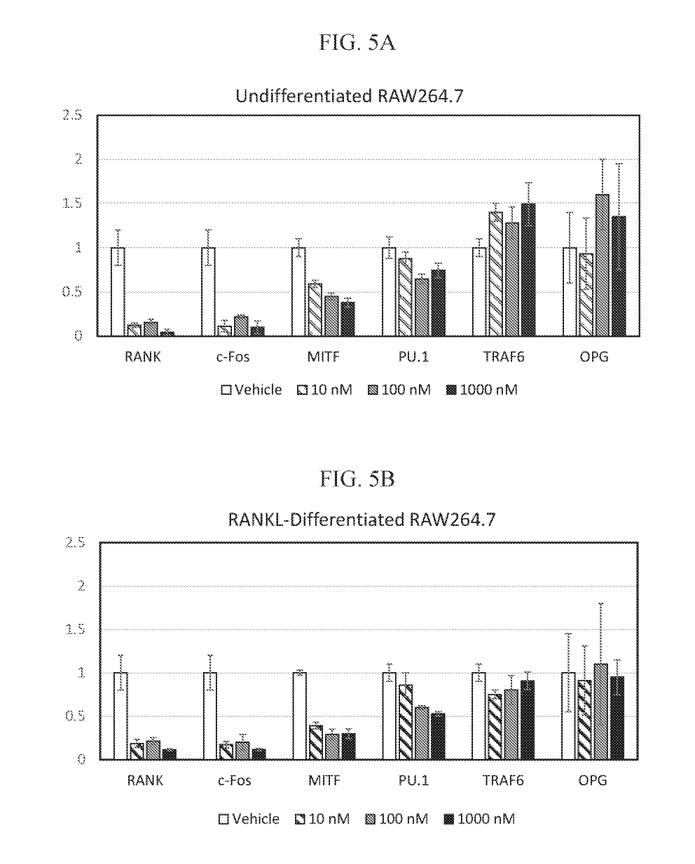

Finally, apilimod potently blocked RANKL-stimulated osteoclastogenesis of RAW264.7 macrophages in vitro (see FIG. 3 and FIGS. 4A-4B). Apilimod blocked the expression of RANK receptor and the transcription factors MITF, PU.1 and c-Fos in both undifferentiated and RANKL-differentiated RAW264.7 macrophages. Cells were differentiated for a total of 3 days with 30 ng/mL RANKL. On the last day of differentiation cells were co-treated with RANKL and the indicated concentration of apilimod or vehicle for 24h. Decreases were observed for the osteogenic factors RANK, c-Fos, MITF, and PU.1. Significant decreases were not observed for the osteogenic factor TRAF6 or the anti-osteogenic factor OPG. (see FIGS. 5A-5B).

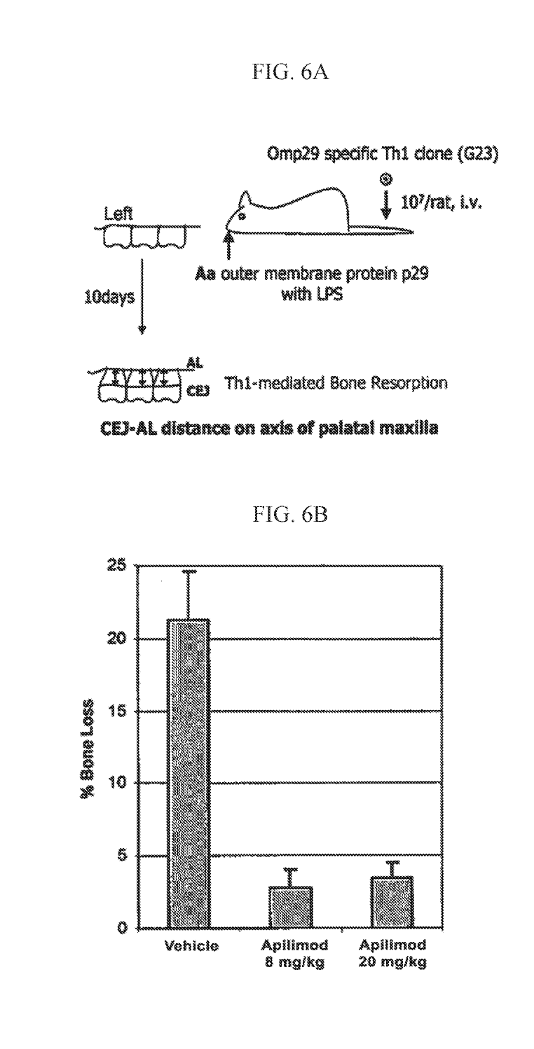

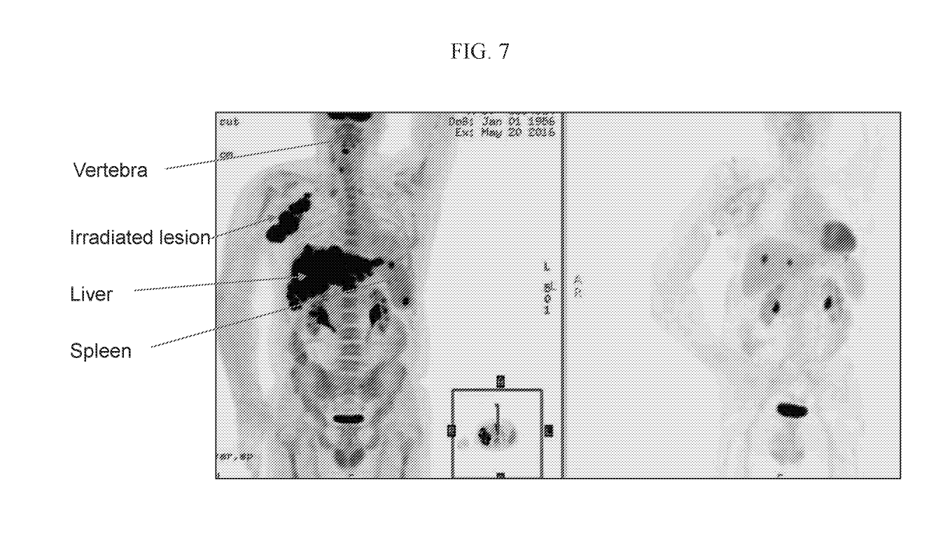

Moreover, apilimod was active in inhibiting in vivo osteoclast activity in a rat periodontal disease model (see FIGS. 6A-6B). In brief, Th-1 type clonal cells specific for Actinobacillus actinomycetemcomitans (Aa) 29-kDa outer membrane protein (Omp29) were activated by incubation with formalin-killed Aa and irradiated syngeneic rat spleen cells. These activated cells were then transferred intravenously through the rat tail vein (1.0×10⁷ cells) into the rat. On the same day, the antigen Omp29 was injected with LPS into the left palatal maxilla gingiva; and saline was injected into the right palatal maxilla gingiva, for control measurements. Apilimod was given orally in daily doses of 8 and 20 mg/kg from the day of the induction until day 10. At the end of the ten day period, animals were sacrificed and their jaws were defleshed to allow the assessment of periodontal bone resorption, calculated as the ratio of the difference in cemento-enamel junction (CEJ-AL) distance between left and right sides versus CEJ-AL distance of right. Both doses of apilimod provided significant protection against Th1-mediated bone loss.

These data indicate that apilimod-induced alterations in cytokine profiles and endolysosomal dynamics block the differentiation of osteoclast precursors as well as block resorptive activity of mature osteoclasts.

### Example 2: Case Study: Metastases of Refractory Lymphoma are Responsive to Apilimod Therapy

A patient with diffuse large B cell lymphoma who had experienced minimal or no response to 7 prior chemotherapies was treated with apilimod dimesylate (see FIG. 7). A PET-CT scan was performed at baseline (left) and then the patient was then treated with 100 mg apilimod dimesylate BID for 6 weeks and a follow up scan was performed 2 weeks later (right). Substantial systemic response was observed in the liver, spleen, and bone (C4 vertebra). Note that the right axillary lymphadenopathy required local radiation therapy before the follow up scan.

This case study supports the use of apilimod dimesylate in treating cancer metastases, including those that are refractory to first-line therapies.

### Example 3: Apilimod Impairs Myeloma Cell Growth in Bone in MPC-1 Syngeneic Model

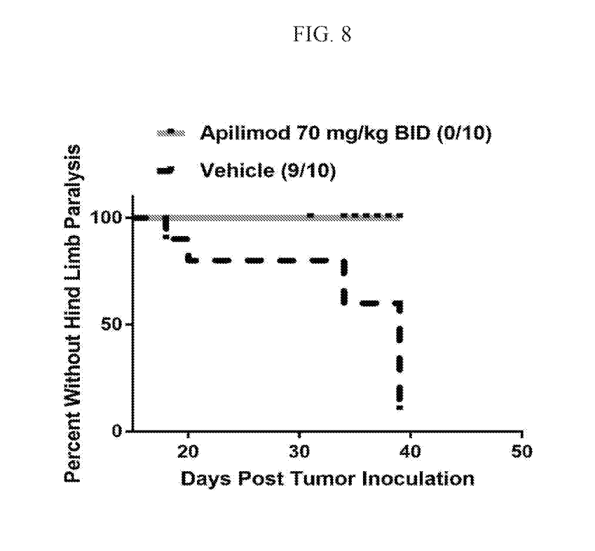

To determine if apilimod inhibition of RANKL/RANK signaling blocks the bone growth of multiple myeloma cells in vivo, the systemic MPC-11 syngeneic model was used (Laskov R et al. J Exp Med. 1970 131(3):515-41; Ferguson V L et al. Bone. 2002 30(1): 109-116). In this model, animals develop hind limb paralysis due to infiltration of multiple myeloma cells into the vertebrae that compresses the spinal column. In brief, MPC-11 tumor cells were maintained in vitro in DMEM medium supplemented with 10% horse serum at 37° C. in 5% CO₂. Each mouse was then injected via the tail vein with MPC-11 tumor cells (1×10⁶) in 0.1 ml of PBS for tumor development. The regimen of dosing is detailed in Table 1. At 4 days post-tumor inoculation, mice were orally administered with a vehicle or apilimod dimesylate (70 mg/kg) twice a day up to 35 days. Survival rate of vehicle-treated animal group (n=10) and apilimod-treated animal group (n=10) were monitored as an indicator of hind limb paralysis. 9/10 animals in the vehicle group were observed to succumb to hind limb paralysis whereas none of the apilimod treated animals displayed this phenotype, suggesting that apilimod impaired the growth of MPC-11 cells in bone. FIG. 8 shows the percentage of surviving animals without hindlimb paralysis. Vehicle group is displayed as a hashed line and the apilimod dimesylate (70 mg/kg BID) group is displayed as a gray solid line (dots indicates removal of animal from experiment due to event unrelated to hind limb paralysis).

TABLE 1
Dosing Regimen of BALB/c MPC-11 systemic syngeneic model
experiment.

| | | | Treatment | | | |
|---|---|---|---|---|---|---|
| | | Tumor cell | (4 days post | | | |
| | | Inoculum | tumor | Dose | Dosing | |
| Group | N | (i.v.) | inoculation) | (mg/kg) | Route | Schedule |
| 1 | 10 | MPC-11 | Vehicle | — | p.o. | BID × 15 |
| | | 1 × 10⁶/ | | | | days |
| 2 | 10 | mouse | Apilimod | 70 | p.o. | BID × 35 |
| | | (day0) | dimesylate | | | days |

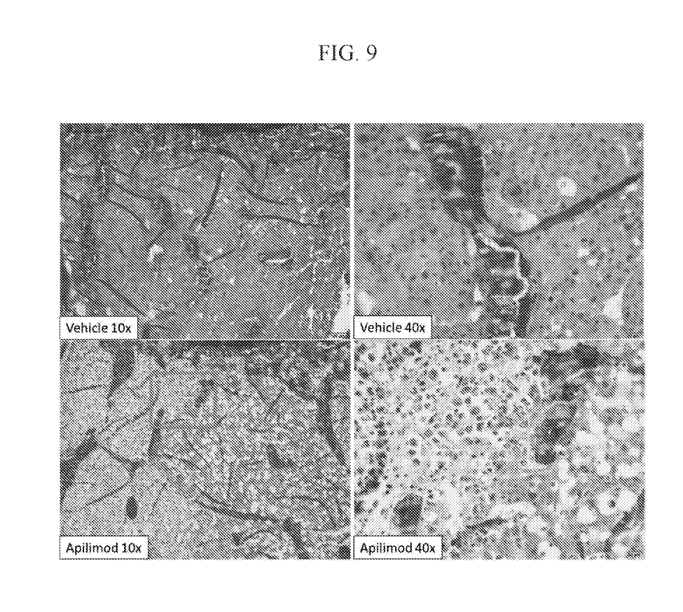

To directly examine the effect of apilimod on the MPC-11 tumor burden in bone, immunohistochemical staining was performed. Femurs from the in vivo experiment were fixed in 10% formalin and then rinsed in 70% ethanol and dehydrated in acetone before embedding in methacrylate. 4 μm sections were then stained in 2% Toluidine blue. FIG. 9 shows representative sections of toluidine blue staining in femurs for vehicle (top) and apilimod (bottom) treated animals at the indicated magnification (10x or 40x). Orange boxed region on left panel is shown at 40× magnification in the right panel. Note total effacement of bone marrow architecture and replacement with MPC-11 tumor cells in top panel. Qualitative analysis revealed massive invasion of myeloma cells with essentially no normal marrow in the vehicle animals compared with minimal invasion observed in apilimod treated animals. This effect was quantitated by generating paraffin sections from tibias of the animals and staining the sections with the multiple myeloma marker CD138 (BioLegend antibody, Clone 281-2). Values for CD138 positive staining were obtained by measuring within in a pre-defined Region of Interest (ROI) defined as 1.17 millimeter square, 200 micrometer below the tibia proximal growth plate. CD138 positive staining was measured according to standard protocols (Dempster D W et al. J Clin Endocrinol Metab. 2012 97(8):2799-2808). The quantitation of the CD138 staining (see Table 2) revealed a significant decrease in bone tumor burden in the apilimod treated group.

TABLE 2
Quantification of CD138 staining

| Parameter | Vehicle (n = 5) | Apilimod (n = 5) | p-value (t-test) |
|---|---|---|---|
| Tumor Area (mm²) | 0.291 ± 0.22 | 0.012 ± 0.03 | 0.048* |
| Tumor Area/ | 0.250 ± 0.19 | 0.010 ± 0.02 | 0.048* |
| Tissue Area (%) | | | |

*represents the p value of statistically significance (p < 0.05).

Together, these data indicate that apilimod inhibits the growth of myeloma cells in bone and reduces bone tumor burden.

1. A method for treating a bone loss associated disease or disorder in a subject in need thereof, the method comprising administering to the subject a pharmaceutical composition comprising a PIKfyve inhibitor selected from apilimod, APY0201, and YM-201636, and pharmaceutically acceptable salts thereof.

2. The method of claim 1, wherein the subject in need is one diagnosed with a disease or disorder selected from the group consisting of hypercalcemia of malignancy, bone metastasis of the breast, bone metastasis of the prostate, cancer treatment induced bone loss, multiple myeloma, rheumatoid arthritis, psoriastic arthritis, osteoporosis, skeletal unloading or disuse, sporadic Paget's disease, juvenile Paget's disease, thyrosine excess and hyperthyroidism, periprothetic bone loss, periodontal disease, and cancer metastasis.

3. The method of claim 1, wherein the PIKfyve inhibitor is apilimod free base or apilimod dimesylate.

4. The method of claim 3, wherein the PIKfyve inhibitor is apilimod dimesylate, and the amount of apilimod dimesylate in the composition is from about 0.001 mg/kg to about 1000 mg/kg.

5. The method of claim 1, further comprising administering to the subject an anti-resorptive agent or anti-RANKL agent, or a combination thereof.

6. The method of claim 5, wherein the anti-resorptive agent is selected from the group consisting of progestins, polyphosphonates, bisphosphonate(s), estrogen agonists, estrogen antagonists, estrogen, estrogen derivatives, and combinations thereof.

7. A method for treating a metastasis of a primary cancer in a subject in need thereof, the method comprising administering to the subject a pharmaceutical composition comprising at least one PIKfyve inhibitor selected from apilimod, APY0201, and YM-201636, and pharmaceutically acceptable salts thereof.

8. The method of claim 7, wherein the primary cancer is selected from the group consisting of lymphoma, multiple myeloma, breast cancer and prostate cancer.

9. The method of claim 7, wherein the metastasis is a bone metastasis.

10. The method of claim 7, wherein the primary cancer is multiple myeloma and the metastasis is a bone metastasis.

11. The method of claim 7, wherein the metastasis is refractory to standard first line therapy.

12. The method of claim 7, wherein the PIKfyve inhibitor is selected from apilimod free base and apilimod dimesylate.

13. The method of claim 12, wherein the PIKfyve inhibitor is apilimod dimesylate and the amount is from about 0.001 mg/kg to about 1000 mg/kg.

14. The method of claim 13, further comprising administering to the subject at least one additional therapeutically active agent selected from the group consisting of an anti-CTLA4 antibody, an anti-PD-1 agent, an anti-PD-L1 agent, and an anti-PD-L2 agent.

15. The method of claim 14, wherein the at least one additional therapeutically active agent is an anti-PD-1 antibody or the anti-CTLA4 antibody, ipilimumab.

16. A method for treating giant cell tumor of bone (GCTB) in a subject in need thereof, the method comprising administering to the subject a pharmaceutical composition comprising a PIKfyve inhibitor selected from apilimod, APY0201, and YM-201636, and pharmaceutically acceptable salts thereof.

17. The method of claim 16, wherein the PIKfyve inhibitor is apilimod dimesylate.

18. The method of claim 17, wherein the amount of the apilimod dimesylate is from about 0.001 mg/kg to about 1000 mg/kg.

19. The method of claim 17, further comprising administering to the subject at least one additional therapeutically active agent selected from the group consisting of an anti-RANKL agent, an anti-CTLA4 antibody, an anti-PD-1 agent, an anti-PD-L1 agent, and an anti-PD-L2 agent, and combinations thereof.

20. The method of claim 19, wherein the at least one additional therapeutically active agent is selected from an anti-PD-1 antibody, the anti-CTLA4 antibody, ipilimumab, and the anti-RANKL agent, denosumab.

21. A method for treating multiple myeloma in a subject in need thereof, the method comprising administering to the subject a pharmaceutical composition comprising at least one PIKfyve inhibitor selected from apilimod, APY0201, and YM-201636, and pharmaceutically acceptable salts thereof.

22. The method of claim 21, wherein the at least one PIKfyve inhibitor is apilimod dimesylate.

23. A pharmaceutical pack or kit comprising, in separate containers or in a single container, a unit dose of at least one PIKfyve inhibitor selected from the group consisting of apilimod, APY0201, and YM-201636, and pharmaceutically acceptable salts thereof, and a unit dose of at least one additional agent.

24. The pharmaceutical pack or kit of claim 23, wherein the at least one additional agent comprises an anti-resorptive agent or anti-RANKL agent, or a combination thereof.

25. The pharmaceutical pack or kit of claim 24, wherein the anti-resorptive agent is selected from the group consisting of progestins, polyphosphonates, bisphosphonate(s), estrogen agonists, estrogen antagonists, estrogen, estrogen derivatives and combinations thereof.
